(19) United States
(12) Patent Application Publication
(10) Pub. No.: US 2023/0107532 A1
(43) Pub. Date: Apr. 6, 2023

(54) METHOD AND SYSTEM FOR MONITORING EFFECTS OF HEALTH AND LIFESTYLE INTERVENTION

(71) Applicant: OKEN SANTE, Sherbrooke (CA)

(72) Inventors: Alan Cohen, New York, NY (US); Frederik Dufour, Sherbrooke (CA); Alexandre Lajoie, Quebec (CA); Pascal Azadian, Vicheres (FR)

(21) Appl. No.: 17/930,579

(22) Filed: Sep. 8, 2022

Related U.S. Application Data
(60) Provisional application No. 63/261,006, filed on Sep. 8, 2021.

(51) Int. Cl. G16H 20/00; G16H 50/20

(52) U.S. Cl. CPC G16H 20/00; G16H 50/20

(57) ABSTRACT

A method and system for health and lifestyle interventions is provided, the system comprising three modules: an evaluation module, a suggestion module and a user interface module. The evaluation module uses rigorous statistical comparison of the levels before and after the start of the intervention and the suggestion module guides the individual through the process by the user interface module.

### CROSS REFERENCE TO RELATED APPLICATIONS

The present patent application claims the benefits of priority of U.S. Provisional Pat. Application No. 63/261,006, entitled “ METHOD AND SYSTEM FOR MONITORING EFFECTS OF HEALTH AND LIFESTYLE INTERVENTION”, and filed at the U.S. Pat. and Trademark Office on Sep. 8, 2021, the content of which is incorporated herein by reference.

### FIELD OF THE INVENTION

The present invention generally relates to the field of methods and system for monitoring effects of health and lifestyle interventions of users. More specifically, the present invention relates to computer-implemented methods and systems to improve health of users through monitoring of effects of health and lifestyle practiced by users.

### BACKGROUND OF THE INVENTION

Many, if not most, individuals wish to improve their health. Lifestyle interventions such as diets, exercise programs, supplements, meditation, and better sleep habits are well recognized as some of the most effective ways to improve one’s health. However, not all lifestyle interventions are effective, and not all interventions work for each person.

Currently, individuals practicing lifestyle interventions to improve their health choose interventions in many ways: by reading magazines or online recommendations, by consulting with a health professional, by consulting with trainers or other wellness professionals, by discussing with friends and family, and by trial and error. Some individuals may also consult with an increasing number of online platforms that offer artificial intelligence-based recommendations for what interventions they should try. The individuals then try to assess whether the interventions are effective, usually based on how they feel (subjective measures), but sometimes also based on objective measures such as weight, cholesterol levels, blood pressure, or other biomarkers.

This current model of choosing and evaluating health interventions has several key problems. At the level of how interventions are chosen, what has worked for others, or works on average in a population, may not work for a given individual. Each person has a slightly different biology. Targeting the interventions to individual profiles is a step in the right direction, but efficacy still needs to be evaluated.

At the level of how interventions are evaluated, there are several challenges. First, subjective measures of efficacy are sometimes reliable or important (e.g., feeling well rested after a sleep intervention), but others are much harder to evaluate. Is a nutrition intervention effective? Is it the best possible? However, the objective measures also suffer from problems. For example, while it is well known that obesity increases the risk of metabolic disease such as diabetes, not all obese individuals are diabetic, and it is also recognized that there is a subset of healthy obese. For such individuals, trying too hard to lose weight could even be harmful: many extreme diets could come with risks of destabilizing metabolism, even if they are effective for weight loss. Likewise, biomarkers such as cholesterol are part of the overall picture of health, but optimizing them one at a time could, in some circumstances, prove counterproductive. For example, high HDL (high-density lipoprotein) is usually thought to be good for heart disease risk, but in some pre-industrial cultures there are very low levels of HDL and no heart disease, showing that the interpretation of each marker is context dependent.

To add to the challenges, there are too many biomarkers for individuals to simultaneously attempt to optimize all of them. For example, a standard blood and metabolic panel may include around 40 biomarkers, each of which is expected to fall into a certain range. An individual who wants to optimize biomarker profiles by changing lifestyle habits would have a major challenge if interventions that are good for one are bad for another, as can be the case.

Accordingly, solutions are needed to help individuals find promising lifestyle interventions and then assess what interventions work for them. Such a solution would have applications for individual consumers, but also for gyms, nutrition centers, lifestyle medicine, and corporate wellness programs, among others.

A potential solution is provided by global measures of health that integrate numerous clinical biomarkers to generate a composite health score, many of which are published in the scientific literature. Examples of such scores include homeostatic dysregulation as measured by Mahalanobis distance and Klemera-Doubal Biological Age. However, the presence of such a score is not enough: a process is still needed to help individuals measure such a score at multiple time points and evaluate whether the intervention was effective, statistically speaking. Simple comparisons of a single measure before and after are insufficient, as the slightest random fluctuation might be perceived as evidence for or against an intervention. There is thus a need for a sufficient solution that must integrate a rigorous statistical comparison of the levels before and after the start of the intervention, and must guide the individual through the process.

### SUMMARY OF THE INVENTION

The aforesaid and other objectives of the present invention are realized by generally providing a system for improving the health and lifestyle of a user. The system may have an evaluation module, a suggestion module and a user interface module.

A method for improving the health and lifestyle of a user may further be provided, the method configured to use a lifestyle intervention system in accordance with the principles of the present invention.

In one aspect of the invention, a computer-implemented method for improving the health and lifestyle of a subject is provided. The method comprises collecting information about one or more baseline health measurements of the subject, collecting information about a health or lifestyle intervention (HLI) which the subject has started after establishing the baseline health measurement, collecting information about one or more follow-up health measurements which occurred after the subject has started the HLI, quantifying health state of the subject at one or more times prior to starting the HLI by applying a health quantifying algorithm based on the collected information about the one or more baseline health measurements, quantifying health state of the subject at one or more times after starting the HLI by applying the health quantifying algorithm based on the collected information about the one or more follow-up health measurements and calculating a statistical comparison between the quantified health states of the subject before and after the start of the HLI to evaluate the efficacy of the HLI.

The method may further comprise the health quantifying algorithm integrating individual characteristics of the subject.

The method may further comprise the calculation of the statistical comparison using one or more statistical models. The statistical models may comprise one or a combination of the following: t-tests, tests for trend, multiple regression and change-point analysis.

The health quantifying algorithm may be selected in one or a combination of the following: DNA methylation age, epigenetic clocks, inflammation scores, biological age scores, Mahalanobis distance, homeostatic dysregulation, integrated albunemia, critical transition risk scores, proteomic age, metabolomic age and integrated -omics age.

The method may further comprise allowing a user to interact with the calculated statistical comparison. The method may further comprise allowing the user to interact with one or a combination of the following: health-related measurements before and after the start of the HLI, data regarding other subjects, data regarding previous HLIs of the subject, other users, data regarding health and lifestyle of the subject, protocols and reminders regarding the HLI and electronic messages.

The method may further comprise suggesting one or more additional HLIs for improving the health and lifestyle of the subject. The suggestion of one or more additional HLIs may be based one or more of the following: data relating to success and/or failure of other subjects, individual preferences or limits for types of HLIs, individual characteristics of the subject or other subjects, health measurements of the subject or other subjects, health quantifying algorithm data of the subject or other subjects and additional information on efficacy and safety of HLIs. The step to suggest one or more additional HLIs may use artificial intelligence. The artificial intelligence may use one or a combination of the following: linear regression, logistic regression, elastic net regression, regression trees and random forests, neural networks, deep learning methods, nearest neighbor approaches and Bayesian statistics.

The method may further comprise allowing a user to interact with the calculated statistical comparison or may further comprise allowing a user to interact with uncertainty of the difference between the quantified health states of the subject before and after the starting of the HLI. The uncertainty may be estimated using any one or a combination of the following: confidence intervals, credibility intervals and p-values. The method may further comprise allowing the user to interact with one or a combination of the following: health-related measurements before and after the start of the HLI, data regarding other subjects, data regarding previous HLIs of the subject, other users, data regarding health and lifestyle of the subject, protocols and reminders regarding the HLI and electronic messages. The method may further comprise allowing a user to interact with the one or more suggested additional HLIs.

In another aspect of the invention, a computer system for improving the health and lifestyle of a subject is provided. The system comprises an evaluation module configured to collect information about one or more baseline health measurements of the subject, to collect information about a health or lifestyle intervention (HLI) which the subject has started after establishing the baseline health measurement, to collect information about one or more follow-up health measurements which occurred after the subject has started the HLI, to quantify health state of the subject at one or more times prior to starting the HLI by applying a health quantifying algorithm based on the collected information about the one or more baseline health measurements, to quantify health state of the subject at one or more times after starting the HLI by applying the health quantifying algorithm based on the collected information about the one or more follow-up health measurements and to calculate a statistical comparison between the quantified health states of the subject before and after the starting of the HLI to evaluate the efficacy of the HLI.

The system may further comprise a user interface module allowing a user to interact with the calculated statistical comparison.

The system may further comprise a suggestion module configured to suggest one or more additional HLIs for improving the health and lifestyle of the subject. The system may further comprise a user interface module allowing a user to interact with the calculated statistical comparison and with the one or more suggested additional HLIs.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is a diagram of a method of health and lifestyle intervention.

FIG. 2 is a diagram of a first embodiment of an evaluation module in accordance with the principles of the present invention.

FIG. 3 is a diagram of a second embodiment of an evaluation module in accordance with the principles of the present invention.

FIG. 4 is a diagram of a first embodiment of a suggestion module in accordance with the principles of the present invention.

FIG. 5 is a diagram of a second embodiment of a suggestion module in accordance with the principles of the present invention.

FIG. 6 is a diagram of a third embodiment of a suggestion module in accordance with the principles of the present invention.

FIG. 7 is a diagram of a first embodiment of a user interface module in accordance with the principles of the present invention.

FIG. 8 is a diagram of a second embodiment of a user interface module in accordance with the principles of the present invention.

### DETAILED DESCRIPTION OF THE INVENTION

A novel method and system for monitoring effects of health and lifestyle interventions will be described hereinafter. Although the invention is described in terms of specific illustrative embodiment(s), it is to be understood that the embodiment(s) described herein are by way of example only and that the scope of the invention is not intended to be limited thereby.

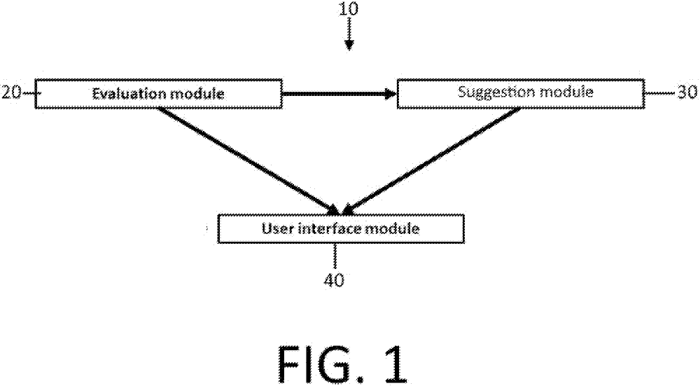

Referring to FIG. 1, an embodiment of a process and of a method for evaluating the efficacy of interventions to improve health, to generate suggestions for future interventions, and to allow users to follow the proper evaluation process and interact effectively with a system 10 in ways that enhance their uptake of effective interventions is illustrated. Accordingly, the system 10 comprises a plurality of modules which may be combined or used individually for different aspects of the process of choosing and evaluating health interventions in subjects. For example, a given subject may already possess all the information necessary to evaluate the efficacy of a past intervention, or may not yet have tried an intervention and may require a suggestion. Accordingly, the modules 20, 30 or 40 may be used singularly or jointly. The modules of the system 10 may comprise an evaluation module 20, a suggestion module 30, and a user interface module 40. The evaluation module 20 generally aims at helping users assess whether an intervention designed to improve a subject’s health is effective, harmful, and/or neutral. The suggestion module 30 generally provides the user with recommended interventions that are likely to improve the health of the subject. The user interface module 40 allows the user/subject to interact with the results of the evaluation module and the suggestion module, as well as additional functionalities. All three modules are described in sequence below. Understandably, the system 10 may comprise additional modules which may be configured to add capabilities to the system 10 or to offer additional functions to the user.

Within the present disclosure, a “subject” generally comprises an organism for whom an improved health state is desired. The subject will usually be a human, but may also be an animal or even a plant. In most cases of human subjects, the subject will be the one following his/her own health and making decisions, but there may be situations when another individual is monitoring the results for a subject. This would obviously be the case for an animal or plant, but could apply in some cases for humans as well, such as for children, persons with dementia, doctors following patients, or employers tracking employee health. The present disclosure refers to the person monitoring the results as the “user”; as noted above, in most cases the subject and the user are the same individual.

### Evaluation Module

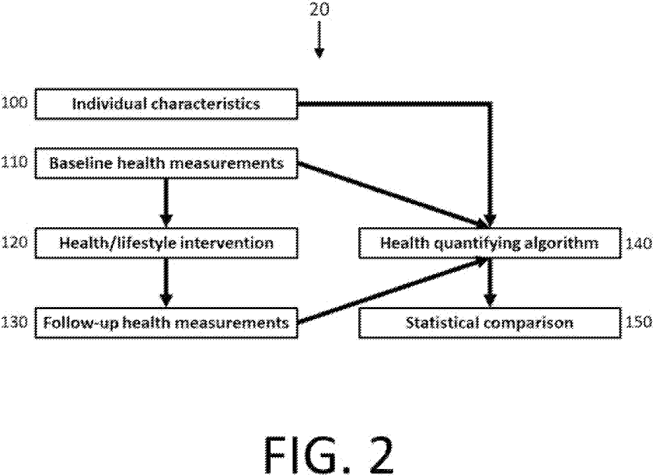

Referring now to FIG. 2, an embodiment of the evaluation module 20 executing a computer-implemented method to evaluate is shown. The evaluation module 20 generally comprises a set of one or more baseline measurements 110. The one or more baseline measurements may be selected from a suite of one or more Health-related Measures (HRMs), which can be used to establish the state of the subject before an intervention. The evaluation module 20 may further use one or more Health or Lifestyle Interventions 120 (HLI), which is generally intended to improve the health state or an aspect of the health state of the subject. The evaluation module 20 further comprises a series of one or more follow-up measurements 130 related to the set of HRMs. The measurements 130 are conducted after the start of the HLI and can be used to compare the state of the subject after the HLI to the state that was established via the baseline measurements 110. The evaluation module 20 is further configured to execute a Health-quantifying Algorithm 140 (HQA). The HQA 140 generally integrates one or more of the HRMs and computes a composite score of health state. The computed score of health state may be a measure of global health, or of an aspect of health (e.g., mental health) that may be affected by the HLI. The evaluation module 20 is further configured to automatically perform a statistical comparison 150 between the baseline measurements 110 and follow-up measurements. Nearly all HRMs are likely to have some level of measurement error and to show some degree of random temporal fluctuation. An appropriate statistical model is thus necessary to evaluate whether any apparent effects of the HLI are real as opposed to due to statistical noise, and to assess the magnitude of any benefit or harm, as well as any uncertainty around the estimate of benefit, harm, or lack of effect. Non-exhaustive examples of statistical models would include t-tests, tests for trend, multiple regression, and change-point analysis. Understandably, other types of statistical models could be used. The evaluation module 20 is further configured to store Individual Characteristics (ICs, 100) describing the subject, a plurality of which may be incorporated into the HQA 140, if needed or appropriate.

The set of baseline measurements 110 may be embodied as a set of multiple measurements that allows the statistical comparison 150 (see below) to arrive at a more precise estimation of any benefit. The multiple measurements may be spaced in time based on known temporal fluctuations. For example, a blood draw might be conducted every week for four weeks in order to generate a set of four weekly profiles, establishing a clear baseline over the month preceding the HLI. However, in some cases a single baseline measurement 110 might be sufficient, for example if there were sufficient complementary data to show sufficient stability and precision of this measure to generate the subsequent estimates needed for a statistical comparison. The HRMs measured are those that are necessary for the HQA 140 (see below).

The HLI 120 can be any intervention with an aim to improve health globally, or in the domain(s) targeted by the HQA 140. For example, a specific diet such as a ketogenic diet, or a specific exercise program such as high-intensity interval training might be expected to affect global physical health, and would be appropriate interventions to test in relation to HQAs 140 drawing on composite biomarkers of health. Alternatively, cognitive behavioral therapy interventions might have effects on global mental health, and could be paired with HRMs and HQAs 140 targeted to measuring mental health. Sometimes, the impact of an intervention could have ripple effects, such that mental health interventions might affect physical health, or vice versa. There are thus few limitations on what types of interventions might be attempted in order to improve different aspects of health. The HLIs are generally clearly temporally defined, so as to permit identification of the baseline measurements 110 before the intervention started and subsequent follow-up measurements 130 after the start of the intervention. The HLI may further be relatively discrete, so as to be distinguishable from other changes or interventions in the subject’s life. For example, a diet intervention that started at the same time as chemotherapy would be hard to separate in terms of its effects; likewise, a combination of diet and exercise would not permit a separate evaluation of the diet component and of the exercise component, unless future tests were conducted on at least one individually. Because many factors change constantly in the lives of subjects, it is not expected that interventions will be perfectly discrete from other changes.

The follow-up measurements 130 are generally embodied as a series of one or more measurements taken after the start of the HLI 120. Depending on the nature of the HLI 120, the follow-up measurements 130 may be taken after either the completion of the HLI 120, or after the start of the HLI 120. For example, if the HLI 120 is a continuous intervention such as a change in diet or taking supplements, it is expected that the HLI 120 would be ongoing during the follow-up measurements 130. On the other hand, a temporally discrete HLI 120 such as a meditation retreat might be expected to have finished before the follow-up measurements 130. In the case of ongoing/continuous HLIs 120, it may be advisable to have a certain delay between the start of the HLI 120 and the first follow-up measurement in order to allow time for the intervention to take effect; such delay typically depends on the nature of the HLI 120 and is generally judged on a case-by-case basis. It can also depend on the statistical model used for comparison 150 (see below). The number of follow-up measurements 130 generally depends on the level of precision of the measures and of the HQA 140, but will often be greater than the number of baseline measurements 110, particularly in embodiments if the statistical comparison 150 attempts to identify a trend of improvement during a continuous HLI 120, as opposed to a new state after a temporally discrete HLI 120. Like for the baseline measurements 110, there may be circumstances in which a single follow-up measurement 130 is sufficient, but generally multiple measurements 130 are expected to be required for an appropriate comparison 150.

In some embodiments, the HQA 140 is an algorithm that integrates one or more HRMs into a generalized score of health or well-being, or into a score that quantifies health or well-being in a specific domain. The algorithm can integrate a single measure temporally, or multiple measures at a single timepoint, or multiple measures at different timepoints. For illustrative purposes only, the HRM may include: (1) heart rate data integrated temporally to generate measures of heart rate variability; (2) multiple blood biomarkers can be integrated with Mahalanobis distance to generate scores of homeostatic dysregulation, either globally, or for specific physiological systems/domains; (3) Klemera-Doubal biological age; (4) questionnaire based depression scores; (5) an integration of multiple measures above to generate a score combining multiple scores. In some cases, the HQA 140 might be a single HRM at a single timepoint, if this is the most efficient way to measure the aspect of health in question. Understandably, in other embodiments, the HRM may comprise other markers or measurements to quantify health and/or well-being of the subject.

The HQA 140 may in some cases use other information beyond the HRMs, such as ICs 100. For example, sex is not an HRM, but may modify how the HRMs predict health. In this case, a separate HQA 140 might be needed for males and females, and thus a more global HQA 140 (i.e., encompassing two sex-specific HQAs 140) may include sex as a variable to choose the sub-HQA 140 appropriate for the subject. An HQA 140 may thus include one or more HRMs, as well as zero or more ICs, in order to arrive at appropriate quantification of the health state of the subject.

The statistical comparison 150 may be embodied in any ways known or developed in the art to integrate the information generated by the HQA 140 for the baseline 110 and follow-up measurements 130 that allows evaluation of the efficacy of the HLI 120 for the subject in question, while taking into account the variability and/or precision of the HRMs and/or HQAs 140. Accordingly, the statistical comparison 150 may generally permit (1) estimation of the “effect size”: how different the health state (as measured by the HQA 140) is before versus during/after the HLI 120; (2) estimation of the uncertainty and/or precision of the effect size (e.g., confidence intervals, credibility intervals, p-values, etc.).

Many types of statistical comparisons 150 may be appropriate depending on the type of data and the context. For illustrative purposes only, and not meant to be an exhaustive list: (1) a student’s t-test could compare a series of baseline measurements 110 of the HQA 140 to a series of follow-measurements of the HQA 140, in a context where a temporally discrete HLI had finished; (2) a change-point regression model could be used to estimate the change in slope of an HQA 140 at the moment when a continuous/ongoing HLI starts; (3) a multi-level model that integrates data of multiple subjects in order to better estimate the temporal variability of the HQAs 140 could be used to arrive at more precise estimates of benefits/harms for a specific subject with a smaller number of baseline 110 and/or follow-up measurements 130; (4) A decay model could be used to estimate how quickly the benefits of a temporally discrete HLI wear off after the intervention.

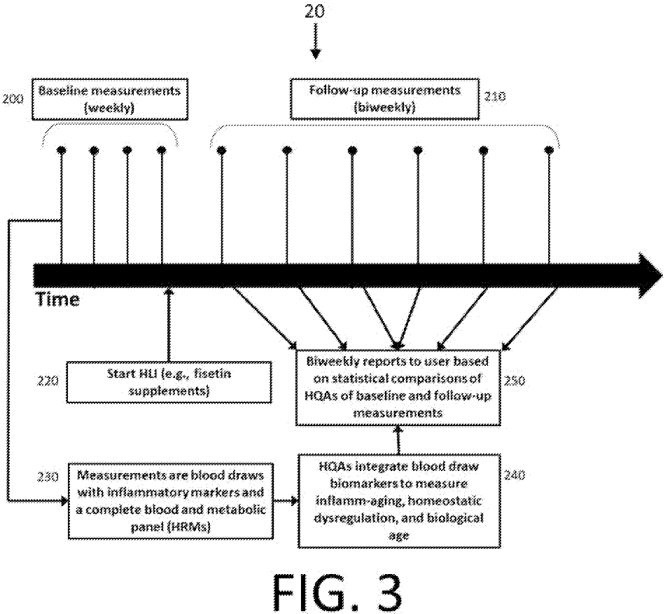

Referring now to FIG. 3 is an exemplary embodiment of the evaluation model 20 illustrated as a timeline. Fisetin is a phytochemical produced in low quantities in strawberries and that has shown senolytic (anti-cellular senescence) effects in laboratory experiments. A subject may wish to improve her global physical health by taking fisetin supplements at doses much higher than in strawberries, thereby eliminating or reducing cellular senescence and improving many aspects of health. However, this treatment is unproven in humans generally, and may comprise risks. The supplement is nonetheless available commercially, and is being used by subjects attempting to slow their aging rates. The proposed method/process cannot replace an epidemiological study that could evaluate potential risks and benefits at a population level (e.g., increased risk of cancer in younger individuals). However, it could help evaluate whether the subject is improving one or more aspects of health in the near term. Cellular senescence is known to cause chronic low-grade inflammation; one prediction is thus that the intervention would reduce chronic low-grade inflammation. An algorithm to quantify chronic low-grade inflammation (“inflamm-aging”) was published by Morissette-Thomas et al. (2014). In the present example, the HQA 140 may use a set of inflammatory markers measured in a blood sample (the HRMs) based on the said study. The HQA 140 may further be configured to measure homeostatic dysregulation globally and in six key physiological systems (oxygen transport, liver/kidney function, micronutrients, hematopoiesis, electrolytes, and lipids) as described by Li et al. (2015) based on 36 standard clinical biomarkers (hemoglobin, albumin, cholesterol, electrolytes, etc., the HRMs). In yet other embodiments, the HQA 140 may further be configured to quantify Klemera-Doubal biological age (2006) using the same set of HRMs as used for homeostatic dysregulation. Understandably, the HQA 140 may be embodied as a single computing module or may be split into a plurality of computing modules.

The execution of the one or more HQAs 140 may produce an advice or counsel to the subject. As an example, the system 10 may compute and generate a counsel for a subject to take four weekly blood tests 200 (corresponding to the baseline measurements 110 in FIG. 2) over a month before starting fisetin supplements, without making any other major changes in lifestyle, but allowing for normal daily variation. Then, fisetin supplements 220 would be taken at a standard dose and regimen (corresponding to the HLI 120 in FIG. 2). After one week, a series of six biweekly blood tests 210 would begin (corresponding to the follow-up measurements 130 in FIG. 2). At each blood test, inflammatory and other biomarkers would be collected, the HRMs, 230. The HQAs 140 at each time point may then be calculated based on the HRMs 230 collected at the respective time points, effectively integrating the HRMs 230 via the HQAs 140. In such an example, the integration allows measuring the general impact of the supplements on inflamm-aging, homeostatic dysregulation, and biological age over a 12-week period. In such an embodiment, after each follow-up measurement 210 is performed, an online platform (see below) or server may be configured to calculate or compute the statistical comparison 150. In the illustrated embodiment, the statistical comparison 150 uses a change-point regression model which assumes a flat slope (no temporal change) for the four baseline measurements 110, and assesses the evidence that the said slope changes (improvement or worsening on each HQA 140) at the moment that the fisetin supplements start to be ingested by the subject. In some embodiments, the online platform or server may be configured to compute a confidence interval around the slope estimate, and a p-value for the estimate of a change in slope (i.e., benefit or harm) 250. The calculations 250 may be displayed to the user using any type of displaying device. At each subsequent follow-up measurement 130, the system 10 may be configured to compute or calculate a new estimate of slope change and precision. In some embodiments, the precision may be increased given the greater quantity of data. In the present example, no change in slope is detected for any HQA 140 after 1, 2, or 3 visits; after the 4th visit, a slight reduction in inflamm-aging becomes clear; after the 5th visit, an important worsening in homeostatic dysregulation becomes apparent for the global score and the liver/kidney score; after the 6th visit, there is still no change in biological age, but a slight worsening in electrolyte dysregulation also becomes apparent, as well as a slight improvement in micronutrient dysregulation. Based on the above exemplary results, the user may then make a decision about whether to continue the supplements based on this information. The process could be conducted with or without the accompaniment of a healthcare or wellness professional.

### Suggestion Module

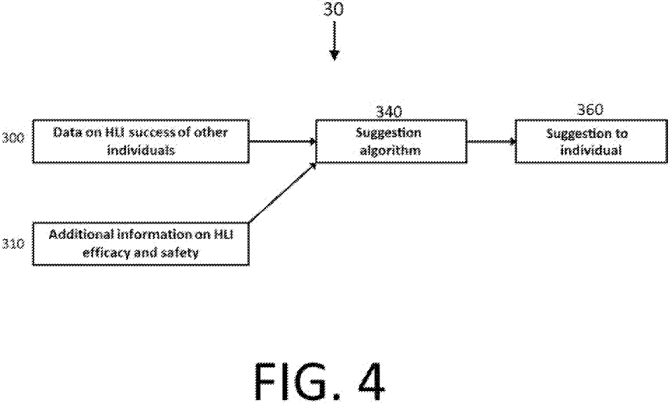

The evaluation module 20 described above is generally applied to a single subject. Now referring to FIGS. 4, 5 and 6, an embodiment of a suggestion module 30 is illustrated. The suggestion module 30 is generally configured to process the testing for a variety of HLIs, or using external data sources and/or information evaluation as performed by the evaluation module 20 on a plurality of subjects. The suggestion module 30 may be configured to use artificial intelligence, machine learning, statistics, or other quantitative approaches to provide personalized suggestions to subjects for HLIs that are likely to work for them based on their ICs, HRMs, HQAs 140, and individually stated preferences 330, as well as on additional information on HLI efficacy 310.

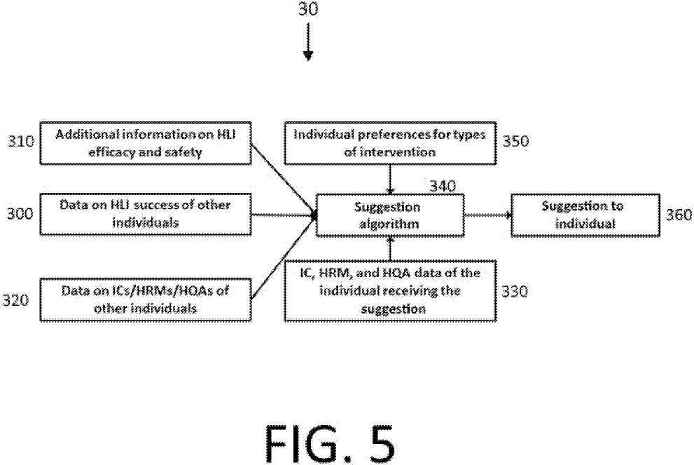

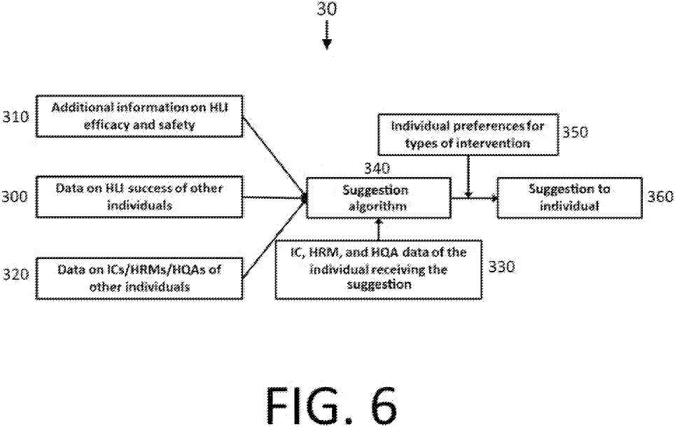

The suggestion module 30, as shown in FIG. 4, FIG. 5, and FIG. 6, generally comprises data on HLI success and/or failure in other subjects, such as but not limited to, a dataset listing different types of HLIs and containing information about how well they have worked to improve the HQA(s) 140 in question 300. The suggestion module 30 may further comprise additional information on HLI efficacy and safety 310 and a suggestion algorithm that quantitatively treats the dataset to evaluate which HLIs work best 340. The suggestion module 30 may further comprise a system for providing suggestions for HLIs to a user 360. The suggestion module 30 may accordingly be embodied as having the four above-recited elements (FIG. 4). Additionally, the suggestion module may further incorporate data on ICs, HRMs or HQAs 140 of subjects in the HLI dataset 320, comparable data on ICs, HRMs, or HQAs in the subject for whom the suggestion is being made 330, and/or preferences or limits of the user receiving the suggestion that could be used to restrict or order the suggestions 350. FIG. 5 represents an embodiment where the preferences or limits of the user 350 are integrated directly into the suggestion algorithm 340 to influence the calculations of the suggestion algorithm 340 accordingly. FIG. 6 represents an embodiment where the preferences or limits of the user 350 are integrated after the suggestion algorithm to filter the results before presentation to the user.

Data on HLI success 300 is likely to be individual-level data, with a structure such as a column including a subject ID code, another column indicating what intervention was attempted, and a third column indicating the success of the intervention. Information on success of the HLI might be qualitative (yes/no it worked or didn’t) or quantitative (how much it worked, with or without uncertainty). Alternative data structures that convey similar information are also covered. Aggregate data (i.e., data summarizing what interventions have been successful with respect to improving the HQA 140 in question, but not containing information on specific individuals) are also covered.

Additional information on HLI efficacy and safety 310 may also be used by the suggestion algorithm 340 other than the data on HLI success 300, the data on ICs, HRMs or HQAs 140 of subjects in the HLI dataset 320, or the data on ICs, HRMs, or HQAs 140 in the subject for whom the suggestion is being made 330. For example, if a study emerged showing that the above-mentioned compound fisetin substantially increases risk of breast cancer, fisetin supplements could be excluded from the list of possible suggestions, or excluded for subjects who are women. As an additional example, a literature survey on the impacts of different types of exercise could be included in the suggestion algorithm as a Bayesian statistical prior.

Referring now to FIG. 4, a simple embodiment of the suggestion module 30 is illustrated. In such embodiment, data on HLI success 300 is treated simply to evaluate which HLIs perform the best in general, across all subjects. The suggestion algorithm 340 may be embodied as a simple comparison of means (t-test or ANOVA), or of success rates (chi-squared), or any other statistical method appropriate for comparing the relative performance of the HLIs. The system 10 is configured to select and present to the user a suggestion of the single best HLI, or a relative ranking of a plurality of HLIs, or a quantitative comparison of HLIs. Additional information 310 could also be incorporated. However, in this embodiment, the suggestion presented is not personalized to the characteristics of the subject receiving the suggestion.

In addition, the suggestion algorithm 340 may be configured to incorporate additional information about the subjects present in the HLI success dataset, such as, but not limited to ICs, HRMs, and HQAs 320, as shown in FIG. 5. In some embodiments, the suggestion algorithm 340 may be configured to improve the performance of the comparison of the HLIs. In other embodiments, the algorithm 340 may be configured to help match the suggestion to the profile of the subject receiving the suggestion. Any set of additional information may be used, including but not limited to ICs such as sex, age, disease status, genotype, or history of attempted HLIs; HRMs such as but not limited to weight, cholesterol level, or epigenetic markers; or HQAs such as depression scores, homeostatic dysregulation scores, or epigenetic age. These data may be cross-sectional or temporally integrated, and may be aggregated or treated in any way necessary to improve their use in the suggestion algorithm.

If these additional data on ICs, HRMs, and/or HQAs are incorporated into suggestion algorithm 340, it is likely that the same or similar data will be included in data on the subject receiving the suggestion 330. For example, if the subject receiving the suggestion is a 56-year-old female with diabetes, the suggestion algorithm 340 may try to identify a best possible HLI for the female subject based on what has worked in other women aged 55-60 with diabetes; accordingly, this information is necessary for the other subjects in the HLI success dataset. The types of information relating to the subject receiving the suggestion may thus be of the same types, and just as varied, as the information on the other subjects in the HLI dataset.

Beyond matching the suggestion to the profile of the subject receiving the suggestion, the suggestion algorithm may further incorporate information on the preferences or limits of the suggestions. For example, the user may wish to receive suggestions specifically for supplements, or may have a physical limitation preventing certain types of exercise. Such preferences and limits can either be incorporated directly into the suggestion algorithm 30, as seen in the embodiment illustrated at FIG. 5, or can be used as a filter to constrain or order the suggestions before they are communicated to the user, as seen in the embodiment 30 illustrated at FIG. 6. Such information on preferences and limits may be used to completely exclude some HLIs from the suggestions, or may be used to weight them or order them so that they emerge higher or lower in a list of potential suggestions.

The suggestion algorithm may be embodied as any quantitative algorithm known in the art, or that may be developed for this purpose, that can be applied to data on which HLIs have worked in order to provide a single choice or ordered list of recommended HLIs for the subject. Many types of algorithms might be applied, including but not limited to linear, logistic, or elastic net regression; regression trees and random forests; neural networks; deep learning; nearest neighbor approaches; Bayesian statistics; or combinations of the above. The output of the algorithm can be a single best recommended HLI for the subject receiving the suggestion, an ordered list of recommended HLIs for the subject receiving the suggestion, and/or a set of zero or more recommended HLIs with quantitative information about how strongly the HLIs are recommended. Generally speaking, the algorithm generally aims at choosing one or more HLIs that are particularly promising for the subject receiving the suggestion based on the characteristics of the said subject, on the characteristics of other subjects that have attempted HLIs, on the success of the different HLIs, and on the preferences or limits of the subject receiving the suggestion.

Once the suggestion or suggestions have been generated, the generated suggestions are communicated to the user (the subject or a relevant third party, e.g., doctor, parent, employer, etc.) either verbally, in writing, or through an electronic means of communication. The suggestion module generally incorporates information from the evaluation module applied to large numbers of individuals in order to provide personalized suggestions.

In a typical example of use of the suggestion module, a woman in her mid-50s with diabetes and high blood pressure is seeking ways to improve her health on a metabolic health score such as a quantitative measure of metabolic syndrome or the Framingham risk score. The woman has not yet tried any HLIs in the context of the invention, and thus has no prior data on what has worked for her. However, she has undergone an initial blood test and thus has information on where she stands with respect to some HRMs and HQAs. She has also entered relevant individual characteristics into the system 10. In such an embodiment, the system 10 may be connected to a dataset of 50,000 prior users who have collectively tried 200 different HLIs. In such example, 2000 of the prior users are also women with diabetes in their mid-50s. The suggestion algorithm may be configured to use a distance metric (e.g., Euclidean distance, Mahalanobis distance) to score the other users in the system based on how similar their overall profile is to that of the target user, such that the 2000 women in their mid-50s with diabetes would get higher scores. The system 10 may then perform or compute an analysis on all 50,000 prior users and identify or fetch which HLIs worked for the prior users. The system 10 is further configured to weight the prior users based on the proximity scores, such that the 2000 women in their mid-50s with diabetes would have more weight in the analysis. In such an example, the target user may have specified that she wants a physical activity intervention, and that she prefers one that requires less than one hour per day. The suggestion algorithm is further configured to generate a list of the top 10 physical activity interventions, scored based on how well they had performed in other subjects to improve metabolic syndrome and/or Framingham risk score. Understandably, in other embodiments, the algorithm may be configured to generate any other types of lists helping to compare the score of the user with scores or average score of the prior users. In the present example, if one intervention that was slightly more than one hour per day had performed particularly well in the matched cohort of the target subject, it might still be listed, but perhaps not at the top, given her stated preference.

### User Interface Module

The system 10 may further comprise a user interface module 40. The interface module 40 generally permits users to interact with the evaluation module 20, the suggestion module 30, and/or any other additional module. The user interface module 40 comprises an electronic interface or display unit. The electronic interface may be configured to access the user’s results based on the output of the evaluation module 20, to access the user’s suggestions based on the output of the suggestion module 30. The electronic interface may further be configured to provide a user with the ability to follow the results of a subject (which may be said user) over time, the ability to compare results to those of other users individually and/or collectively and the ability to browse a web interface, and/or a mobile application interface.

The user interface module 40 may be further configured to engage in interactions with other users of the interface module in a plurality of manners. Engaging in interactions with other users may be performed anonymously, non-anonymously, by chat or text message, by video, by audio, by email, privately, publicly, or by sharing the user profile or a part thereof. The user interface module 40 may further be configured to comprise the possibility to identify other users with similar profiles or interests or the ability to log daily activities. The daily activities may comprise but are not limited to daily steps, water intake, sleep quality, calorie intake and nutrition, physical activity and/or any lifestyle activity that may be of interest to the users. The user interface module 40 may further be configured to offer the ability to set goals, and the ability to send reminders or encouragements through push notifications, emails, SMS or any other types of notifications.

The user interface module 40 may be further configured to access educational content such as, but not limited to, webinars, videos, blog posts, podcasts, and interactive modules., or to access explanatory material to help understand a plurality of the HRMs, the HQAs, the HLIs, the statistical comparison, the evaluation module, the suggestion module, and/or the appropriate interpretation of results therefrom. The user interface module 40 may be further configured for a user to control access to the user’s data and personalize how that data can be used, to secure the data using highly secure technologies such as blockchain, for users to control the security level of their data, and/or for the system to interface with third-party systems, such as those of an employer or a fitness center. The user interface module 40 may be further configured for third parties to extract data relating to clients of the third parties who are users of the system 10, and/or for the third parties to extract aggregate information relating to the users of the third parties, including information on what HLIs work better, and for which clients.

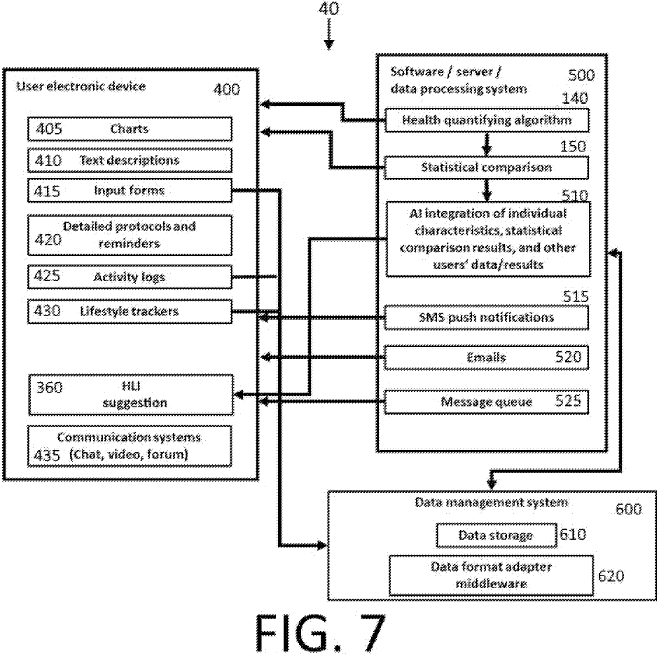

An embodiment of a user interface module 40 is shown in FIG. 7. In such embodiment, the user interface module comprises a user electronic device (400), a server/software/data processing system (SSDPS, 500), and a data management system 600. The user electronic device 400 is typically embodied as a mobile telephone, a computer or a tablet. The electronic device 400 is configured to allow the user to interact with the system 10. The SSDPS 500 is typically configured to manage the interface between user inputs received via the user electronic device 400 and centralized aspects of the system 10 (other users’ data, the user’s historical data, algorithms, etc.), and also processes the data. The SSDPS 500 may be a system hosted on a centralized server. In other embodiments, the SSDPS 500 may be a software installed and being executed by the user electronic device 400, or may be a combination thereof, being thus defined by its functionality rather than its physical location. The data management system 600 comprises a storage unit configured to store, format, secure and/or control access to the data. The storage unit is configured to accept data from the user electronic device 400 and/or from the SSDPS 500. The data management system 600 may be housed on the same server as the SSDPS 500, or separately, and the two may be integrated or not.

The user electronic device 400 offers numerous functionalities to enhance the user’s experience. In some embodiments, the numerous functionalities are provided by an application that is installed on the user electronic device 400. The application may be a mobile application downloadable on the user electronic device 400. The application may thus use the electronic device 400 to show information to users and to receive input from said users and will be in communication with the software/server/data processing system 500 and the data management system 600. The user electronic device 400 may be configured to display charts showing the user’s data and/or other users’ data 405, text descriptions explaining the content 410, input forms 415 permitting the systematic collection of some of the user’s data, detailed protocols and reminders 420 guiding users through HLIs, blood sampling, or other aspects of the process, activity logs 425 that collect data on the users in an automated fashion and send them to the data management system, lifestyle trackers 430 that collect information on the user’s lifestyle and send them to the data management system, HLI suggestions 360 that are generated by the SSDPS 500, and diverse communications systems 435 permitting the user to interact with other users and/or system managers/administrators via formats such as chat, video, forums, etc.

The SSDPS 500 may integrate any past and present data from the subject and other subjects, and may be configured to execute the evaluation module 20 (notably the HQA 140 and the statistical comparison 150) and the suggestion module 30, notably through the use of artificial intelligence 510. The SSDPS 500 may also send push notifications such as SMS 515, emails 520, a message queue 525, etc.

The data management system 600 is typically embodied as a server- or cloud-housed relational database that is enabled to receive data both directly from the user electronic device (activity logs, user input forms, lifestyle trackers, etc.) and from the SSDPS 500 (aggregate indices, past suggestions, etc.). The data can be formatted as appropriate via data format adapter middleware 620. The data is stored 610 and this storage may include appropriate functionalities to ensure security, to personalize access, etc.

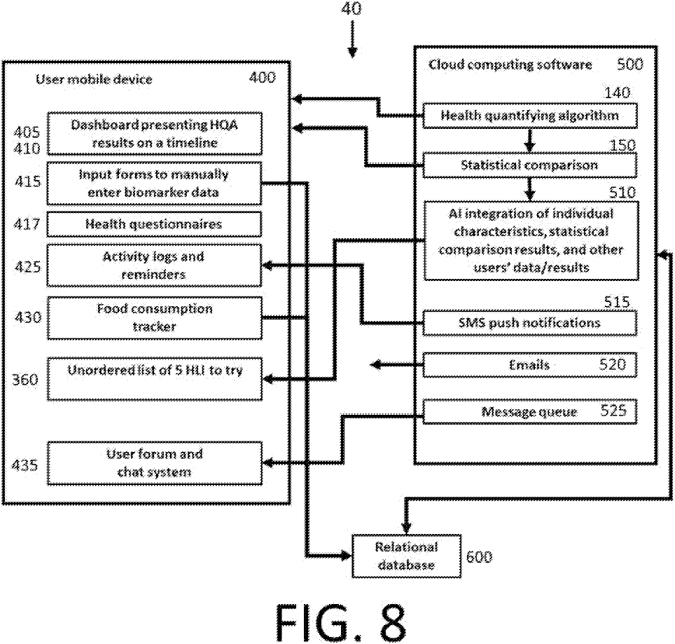

FIG. 8 shows a typical embodiment of the user interface module 40. In this case the user electronic device 400 is embodied as a user mobile device such as a smart phone. An app, not shown, is installed and executed on the device 400. The app is configured to display a dashboard presenting the HQA 140 results as a timeline (405 and 410), input forms to manually enter biomarker data 415, health questionnaires 417, activity logs and reminders 425, a food consumption tracker 430, and a list of suggested HLIs 360 based on the suggestion module 30. The app may further be configured to display or execute a user forum and/or a chat system 435. The SSDPS 500 may be embodied as a cloud computing software/server. In such embodiment, the software 500 is configured to execute the HQA 140, the statistical comparison 150, the suggestion module 30 with AI integration 510, SMS push notifications 515 to the user mobile device, emails 520, and a message queue 525. The data management system 600 may be embodied as a relational database.

The three modules (evaluation 20, suggestion 30, and user interface 40) interact with each other as is shown in FIG. 1. The evaluation module 20 generates results that are fed into the suggestion module 30 as well as stored and displayed via the user interface module 40. The results of the suggestion module are also stored and displayed in the user interface module 40. In some sense, the user interface module 40 is also often an umbrella that may include the functionalities of the evaluation 20 and suggestion 30 modules, since the implementation of the evaluation module 20 and suggestion 30 module will often happen within the user interface module 40. Nonetheless, both the evaluation module 20 and the suggestion module 30 could in theory stand on their own; integration into the user interface module 40 is one embodiment of the present invention.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1. A computer-implemented method for improving the health and lifestyle of a subject, the method comprising:
collecting information about one or more baseline health measurements of the subject;
collecting information about a health or lifestyle intervention (HLI) which the subject has started after establishing the baseline health measurement;
collecting information about one or more follow-up health measurements which occurred after the subject has started the HLI;
quantifying health state of the subject at one or more times prior to starting the HLI by applying a health quantifying algorithm based on the collected information about the one or more baseline health measurements;
quantifying health state of the subject at one or more times after starting the HLI by applying the health quantifying algorithm based on the collected information about the one or more follow-up health measurements; and
calculating a statistical comparison between the quantified health states of the subject before and after the start of the HLI to evaluate the efficacy of the HLI.

2. The method of claim 1 further comprising the health quantifying algorithm integrating individual characteristics of the subject.

3. The method of claim 1, the calculation of the statistical comparison using one or more statistical models.

4. The method of claim 3, the statistical models comprising one or a combination of the following: t-tests, tests for trend, multiple regression and change-point analysis.

5. The method of claim 1, the health quantifying algorithm being selected in one or a combination of the following: DNA methylation age, epigenetic clocks, inflammation scores, biological age scores, Mahalanobis distance, homeostatic dysregulation, integrated albunemia, critical transition risk scores, proteomic age, metabolomic age and integrated -omics age.

6. The method of claim 1 further comprising allowing a user to interact with the calculated statistical comparison.

7. The method of claim 6 further comprising allowing the user to interact with one or a combination of the following: health-related measurements before and after the start of the HLI, data regarding other subjects, data regarding previous HLIs of the subject, other users, data regarding health and lifestyle of the subject, protocols and reminders regarding the HLI and electronic messages.

8. The method of claim 1 further comprising suggesting one or more additional HLIs for improving the health and lifestyle of the subject.

9. The method of claim 8, the suggestion of one or more additional HLIs being based one or more of the following:
data relating to success and/or failure of other subjects;
individual preferences or limits for types of HLIs;
individual characteristics of the subject or other subjects;
health measurements of the subject or other subjects;
health quantifying algorithm data of the subject or other subjects; and
additional information on efficacy and safety of HLIs.

10. The method of claim 9, wherein the step to suggest one or more additional HLIs uses artificial intelligence.

11. The method of claim 10, the artificial intelligence using one or a combination of the following: linear regression, logistic regression, elastic net regression, regression trees and random forests, neural networks, deep learning methods, nearest neighbor approaches and Bayesian statistics.

12. The method of claim 8 further comprising allowing a user to interact with the calculated statistical comparison.

13. The method of claim 12 further comprising allowing a user to interact with uncertainty of the difference between the quantified health states of the subject before and after the starting of the HLI.

14. The method of claim 13, the uncertainty being estimated using any one or a combination of the following: confidence intervals, credibility intervals and p-values.

15. The method of claim 12 further comprising allowing the user to interact with one or a combination of the following: health-related measurements before and after the start of the HLI, data regarding other subjects, data regarding previous HLIs of the subject, other users, data regarding health and lifestyle of the subject, protocols and reminders regarding the HLI and electronic messages.

16. The method of claim 8 further comprising allowing a user to interact with the one or more suggested additional HLIs.

17. A computer system for improving the health and lifestyle of a subject, the system comprising:
an evaluation module configured to:
collect information about one or more baseline health measurements of the subject;
collect information about a health or lifestyle intervention (HLI) which the subject has started after establishing the baseline health measurement;
collect information about one or more follow-up health measurements which occurred after the subject has started the HLI;
quantify health state of the subject at one or more times prior to starting the HLI by applying a health quantifying algorithm based on the collected information about the one or more baseline health measurements;
quantify health state of the subject at one or more times after starting the HLI by applying the health quantifying algorithm based on the collected information about the one or more follow-up health measurements; and
calculate a statistical comparison between the quantified health states of the subject before and after the starting of the HLI to evaluate the efficacy of the HLI.

18. The system of claim 17 further comprising a user interface module allowing a user to interact with the calculated statistical comparison.

19. The system of claim 17 further comprising a suggestion module configured to suggest one or more additional HLIs for improving the health and lifestyle of the subject.

20. The system of claim 19 further comprising a user interface module allowing a user to interact with the calculated statistical comparison and with the one or more suggested additional HLIs.
